(19) United States
(12) Patent Application Publication
(10) Pub. No.: US 2014/0031710 A1
(43) Pub. Date: Jan. 30, 2014

(54) APPARATUS FOR DETERMINING SLEEP APNEA

(71) Applicants: NIHON KOHDEN CORPORATION, Tokyo (JP); WASEDA UNIVERSITY, Tokyo (JP); TOKAI UNIVERSITY EDUCATIONAL SYSTEM, Tokyo (JP)

(72) Inventors: Muneshige KURAHASHI, Tokyo (JP); Naofumi Kobayashi, Tokyo (JP); Sunao Takeda, Tokyo (JP); Takashi Nagaoka, Tokyo (JP); Tadashi Abe, Kanagawa (JP); Tetsuya Urano, Kanagawa (JP); Takuya Aoki, Kanagawa (JP); Chizuko Tsuji, Kanagawa (JP)

(73) Assignees: NIHON KOHDEN CORPORATION, Tokyo (JP); TOKAI UNIVERSITY EDUCATIONAL SYSTEM, Tokyo (JP); WASEDA UNIVERSITY, Tokyo (JP)

(21) Appl. No.: 13/787,928

(22) Filed: Mar. 7, 2013

(30) Foreign Application Priority Data
Jul. 25, 2012 (JP) 2012-164532

(51) Int. Cl. A61B 5/00

(52) U.S. Cl. CPC A61B 5/4818
USPC 600/532

(57) ABSTRACT

An apparatus for determining sleep apnea includes: an expiratory gas collector which is configured to collect an expiratory gas of a subject; an analyzer which is configured to analyze a constituent and a concentration of the constituent in the collected expiratory gas; and a determiner which is configured to determine whether the subject is in sleep apnea or not, based on a concentration of at least VOC in the analyzed expiratory gas.

### CROSS-REFERENCE TO RELATED APPLICATION(S)

This application is based upon and claims the benefit of priority from prior Japanese patent application No. 2012-164532, filed on Jul. 25, 2012, the entire contents of which are incorporated herein by reference.

### BACKGROUND

The presently disclosed subject matter relates to an apparatus for determining sleep apnea in which burdens on the examiner and the subject can be reduced.

As the related art of detecting sleep apnea, there are an apparatus which detects sleep apnea by means of sound (see JP-A-2009-219713), and also an apparatus which detects sleep apnea by using a temperature sensor, a light beam sensor, and an acceleration sensor (see JP-A-2006-320731).

In related-art detection of sleep apnea, however, examinations are often conducted with spending a long time such as all night, and therefore a very large burden is imposed on the subject. Depending on the subject, there is a case where he/she cannot adequately sleep from nervousness, and the examination itself fails. In a measurement in which many sensors are used as disclosed in JP-A-2006-320731, moreover, there is a case where adequate detection is not performed. Consequently, for example, an additional examiner is required for monitoring the detection condition, thereby causing a problem in that a large burden is imposed not only on the subject but also on the examiner's side.

### SUMMARY

The presently disclosed subject matter may provide an apparatus for determining sleep apnea in which the measurement time can be shortened without causing the examination to fail, the measurement timing is not limited to the night, and therefore burdens on the examiner and the subject can be reduced.

The apparatus for determining sleep apnea may comprise: an expiratory gas collector which is configured to collect an expiratory gas of a subject; an analyzer which is configured to analyze a constituent and a concentration of the constituent in the collected expiratory gas; and a determiner which is configured to determine whether the subject is in sleep apnea or not, based on a concentration of at least VOC in the analyzed expiratory gas.

The VOC may include cyclohexane, hexane, and isoprene.

The determiner may compare the concentration of the at least VOC in the analyzed expiratory gas with a reference value that corresponds to the VOC, thereby determining whether the subject is in sleep apnea or not.

The expiratory gas collector may include: a first trap section which is configured to trap at least VOCs in an atmosphere; a second trap section which is configured to trap at least VOCs from the expiratory gas of the subject; and a switching section which is configured to switch a flow passage to a first passage in which the atmosphere which has passed through the first trap section flows into the subject or a second passage in which the expiratory gas of the subject flows into the second trap section. The analyzer may analyze a constituent and a concentration of the constituent, which is trapped in the second trap section.

### BRIEF DESCRIPTION OF THE DRAWINGS

FIG. 1 is a diagram showing an embodiment of an apparatus for determining sleep apnea of the presently disclosed subject matter.

FIG. 2 is a view showing a comparison of concentrations of VOCs in expirations of SAS patients and healthy persons (Healthy).

FIG. 3 is a view showing results of statistical analyses which are performed on results of measurements of cyclohexane in the embodiment of the apparatus for determining sleep apnea of the presently disclosed subject matter.

FIG. 4 is a view showing results of statistical analyses which are performed on results of measurements of hexane in the embodiment of the apparatus for determining sleep apnea of the presently disclosed subject matter.

FIG. 5 is a view showing results of statistical analyses which are performed on results of measurements of isoprene in the embodiment of the apparatus for determining sleep apnea of the presently disclosed subject matter.

### DETAILED DESCRIPTION OF EXEMPLARY EMBODIMENTS

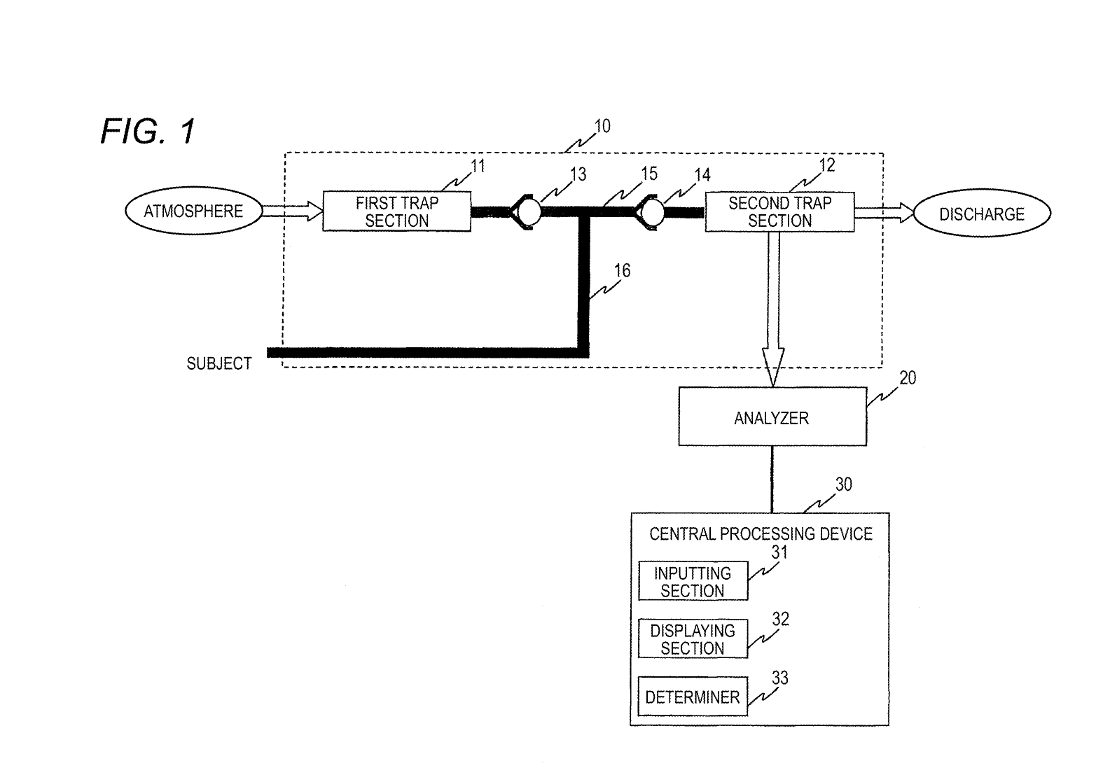

Hereinafter, an embodiment of the apparatus for determining sleep apnea of the presently disclosed subject matter will be described with reference to the accompanying drawings. As shown in FIG. 1, the embodiment of the apparatus for determining sleep apnea includes an expiratory gas collector 10, an analyzer 20, and a central processing device 30.

As the expiratory gas collector 10, the related-art configuration of the purifying/concentrating apparatus disclosed in JP-A-2010-8374 is employed. The expiratory gas collector 10 includes a first trap section 11 and a second trap section 12. The first trap section 11 traps at least VOCs (Volatile Organic Compounds) in the atmosphere. Specifically, a configuration may be employed where an inflow atmospheric gas is cooled to be liquefied or solidified, and at least VOCs are trapped. Alternatively, a configuration may be employed where at least VOCs in an inflow atmospheric gas are adsorbed by an adsorbing material to be trapped. The second trap section 12 has a configuration similar to the first trap section 11, and traps VOCs contained in the expiratory gas of the subject.

A one-way valve 13 is disposed on the output side of the first trap section 11. Another one-way valve 14 is disposed on the input side of the second trap section 12. The one-way valves 13, 14 are connected to each other by a coupling tube 15. A respiratory tube 16 is joined to the coupling tube 15, and a mask which is to be attached to the subject is disposed in an end portion of the respiratory tube 16. The one-way valve 13, the coupling tube 15, the respiratory tube 16, and the one-way valve 14 function as a switching section which controls a flow passage so that the atmosphere which has passed through the first trap section 11 flows into the subject, or so that the expiratory gas of the subject flows into the second trap section 12.

The gas which has passed through the second trap section 12 is discharged to the outside of the expiratory gas collector 10. The analyzer 20 is a gas analyzing apparatus such as a gas chromatograph, and analyzes the constituents and the concentrations of the constituents in the expiratory gas collected in the second trap section 12. Information of the constituents and the concentrations of the constituents in the expiratory gas analyzed by the analyzer 20 is sent from the analyzer 20 to the central processing device 30.

The central processing device 30 is configured by, for example, a computer system, and includes an inputting section 31 such as a keyboard and a mouse, and a displaying section 32 such as an LCD. In the device, a CPU uses software to function as a determiner 33. The central processing device 30 may further include an outputting unit other than the displaying section 32, such as a printer. The determiner 33 determines whether the subject is in sleep apnea or not, based on the concentrations of at least VOCs in the expiratory gas analyzed by the analyzer 20.

Hereinafter, the reason why it is possible to determine whether the subject is in sleep apnea or not, based on the concentrations of VOCs will be described. The inventors have conducted an investigation by using a result of analysis which was performed by the analyzer 20 of the apparatus for determining sleep apnea on patients with the sleep apnea syndrome (SAS) and healthy persons. The analysis was performed on 57 SAS patients and 22 healthy persons by statistical analysis (method: the Mann-Whitney's U-test, significance level of 5%).

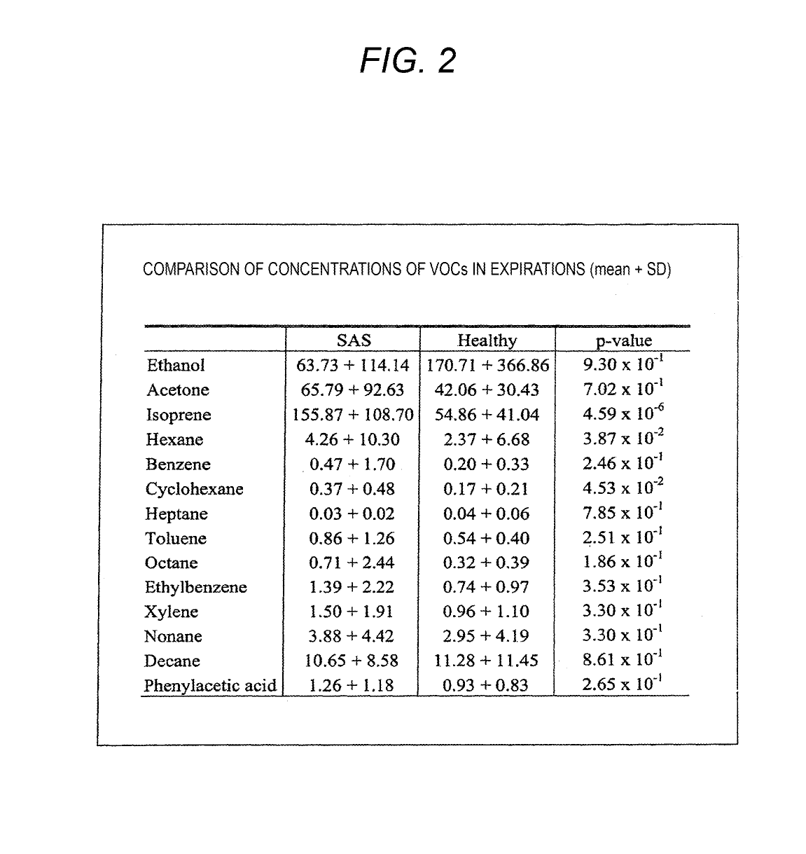

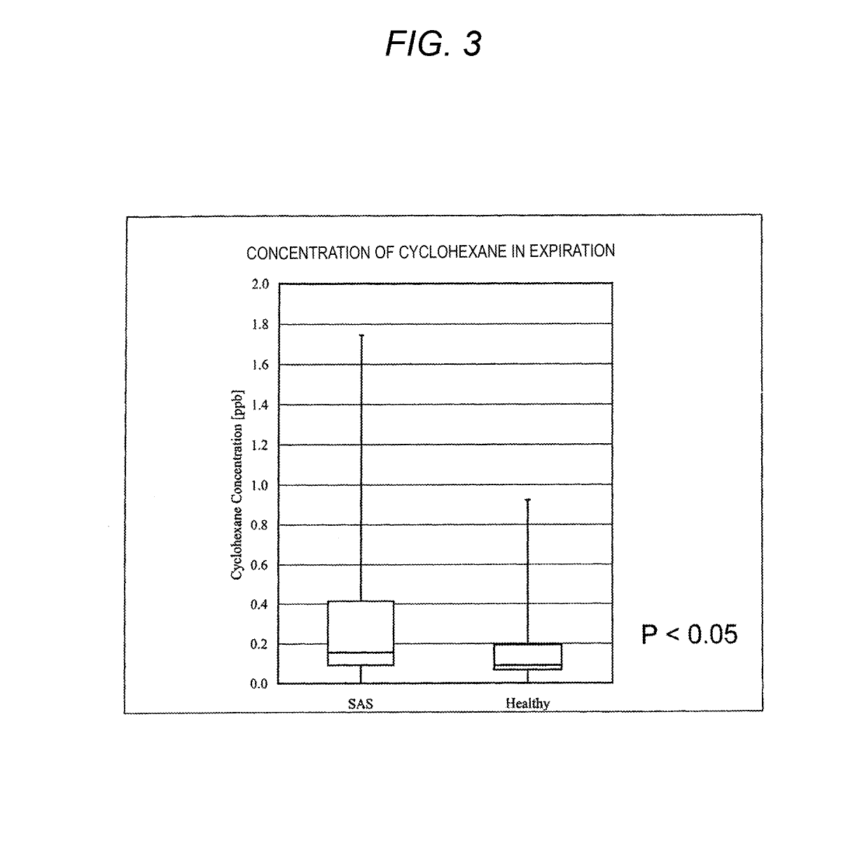

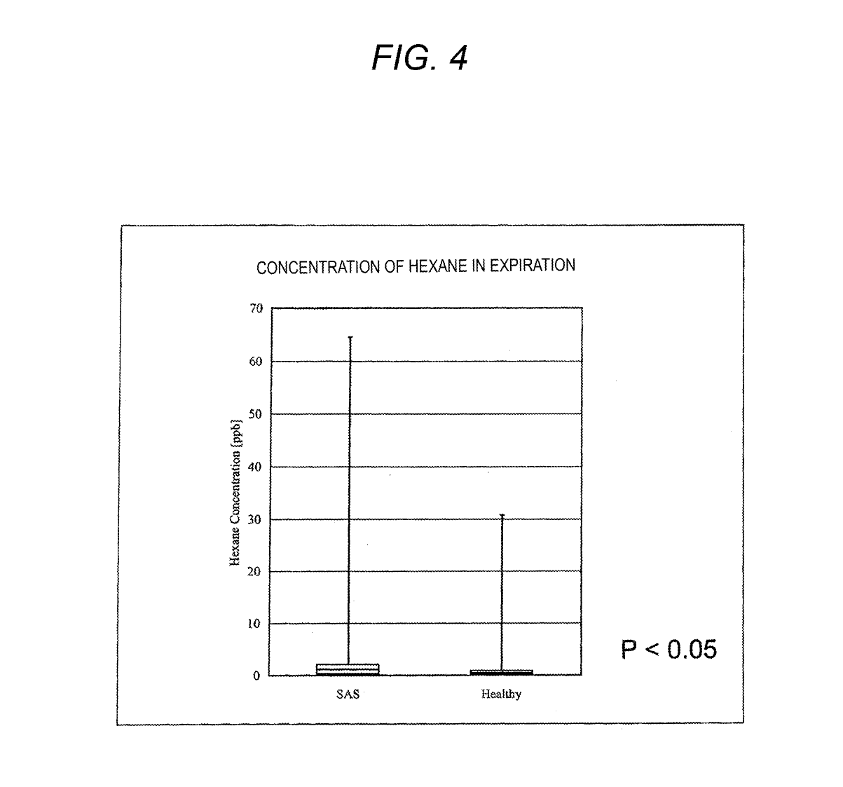

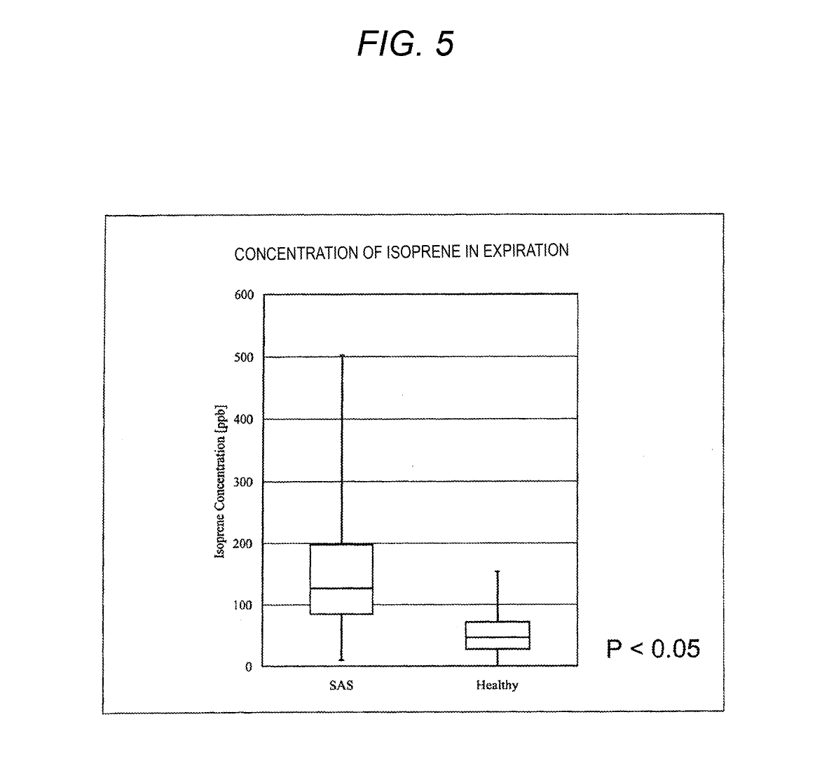

FIG. 2 shows a comparison of VOC concentrations in expirations of the SAS patients and the healthy persons (Healthy) in the above-described measurement. With respect to the concentrations of the VOCs, i.e., cyclohexane, hexane, and isoprene, the concentrations in the SAS patients are higher than those in the healthy persons. FIGS. 3 to 5 show results of the statistical analysis. From the above, it is proper to conclude that the changes of VOCs may possibly be correlated with the pathological condition of the sleep apnea syndrome. Namely, it is possible to determine whether the subject is in sleep apnea or not, based on the concentrations of VOCs.

In the embodiment, the determiner 33 has concentrations of at least VOCs in the analyzed expiratory gas, and information of reference values respectively corresponding to the constituents including VOCs to be used in the determination. The determiner 33 compares the reference values with the concentrations of the analyzed corresponding constituents, and determines whether the subject is in sleep apnea or not. When the investigation is conducted as described above, it is possible to obtain adequate information of a reference value for each constituent. If the concentration is higher (or lower) than the reference value, it is possible to determine that the subject is in sleep apnea. The result of the determination by the determiner 33 is displayed on the displaying section 32, and output from an adequate outputting unit.

For example, the thus configured apparatus for determining sleep apnea performs determination of sleep apnea in the following manner. The apparatus is activated to set a state where trapping of at least VOCs in the atmosphere is enabled in the expiratory gas collector 10. The mask which is not shown is attached to the subject, and the subject breathes, so that the inspiratory gas and the expiratory gas flow through the respiratory tube 16.

At this time, the expiratory gas reaches the second trap section 12 from the respiratory tube 16 through the one-way valve 14, and at least VOCs are trapped in the second trap section 12. The gas which has passed through the second trap section 12 is discharged to the outside. On the other hand, the analyzer 20 analyzes the constituents and the concentrations of the constituents in the expiratory gas collected in the second trap section 12, and information of the constituents and the concentrations of the constituents in the analyzed expiratory gas is sent from the analyzer 20 to the central processing device 30.

In the central processing device 30, the determiner 33 receives the information of the constituents and the concentrations of the constituents in the analyzed expiratory gas, and compares the concentrations of at least VOCs in the analyzed expiratory gas with the information of the reference values respectively corresponding to them. If the concentration is higher (or lower) than the reference value, it is determined that the subject is in sleep apnea. Here, the determination is performed for each of plural constituents of the VOCs. The final determination may be performed by the rule of majority, or under the condition that sleep apnea is determined with respect to all of the objective constituents, that sleep apnea is determined with respect to at least one of the objective constituents, or the like. Particularly, determination whether the subject is in sleep apnea or not may be performed with respect to each of the objective constituents without performing the final determination. Results of these determinations are supplied to the displaying section 32 and the like.

In the above, the embodiment in which the determination is performed for VOCs in a biologically derived gas has been described. Moreover, it is a matter of course that the detection maybe performed by using a constituent which is other than VOCs in a biologically derived gas, and which relates to sleep apnea. In this case, trapping of the constituent, analysis, and determination using corresponding information of a reference value are performed.

According to an aspect of the presently disclosed subject matter, the expiratory gas of the subject is collected, the constituent and the concentration of the constituent in the collected expiratory gas are analyzed, and it is determined whether the subject is in sleep apnea or not, based on the concentration of at least VOC in the analyzed expiratory gas. Consequently, the measurement time can be shortened without causing the examination to fail, the measurement timing is not limited to the night, and therefore burdens on the examiner and the subject can be reduced.

What is claimed is:

1. An apparatus for determining sleep apnea, the apparatus comprising:
an expiratory gas collector which is configured to collect an expiratory gas of a subject;
an analyzer which is configured to analyze a constituent and a concentration of the constituent in the collected expiratory gas; and
a determiner which is configured to determine whether the subject is in sleep apnea or not, based on a concentration of at least VOC in the analyzed expiratory gas.

2. The apparatus according to claim 1, wherein the VOC includes cyclohexane, hexane, and isoprene.

3. The apparatus according to claim 1, wherein the determiner compares the concentration of the at least VOC in the analyzed expiratory gas with a reference value that corresponds to the VOC, thereby determining whether the subject is in sleep apnea or not.

4. The apparatus according to claim 1, wherein
the expiratory gas collector includes:
a first trap section which is configured to trap at least VOCs in an atmosphere;
a second trap section which is configured to trap at least VOCs from the expiratory gas of the subject; and
a switching section which is configured to switch a flow passage to a first passage in which the atmosphere which has passed through the first trap section flows into the subject or a second passage in which the expiratory gas of the subject flows into the second trap section, and
the analyzer analyzes a constituent and a concentration of the constituent, which is trapped in the second trap section.
